(19) United States
(12) Patent Application Publication
(10) Pub. No.: US 2017/0252570 A1
(43) Pub. Date: Sep. 7, 2017

(54) SYSTEMS AND METHODS FOR COMMUNICATION DURING REMOTE PROGRAMMING

(71) Applicant: Boston Scientific Neuromodulation Corporation, Valencia, CA (US)

(72) Inventors: Raul Enrique Serrano Carmona, Los Angeles, CA (US); Chirag Shah, Valencia, CA (US)

(21) Appl. No.: 15/449,751

(22) Filed: Mar. 3, 2017

Related U.S. Application Data
(60) Provisional application No. 62/304,802, filed on Mar. 7, 2016.

(51) Int. Cl. A61N 1/372; G06F 19/00

(52) U.S. Cl. CPC A61N 1/37264; A61N 1/37241; A61N 1/37217; A61N 1/37247; G06F 19/3437

(57) ABSTRACT

A system or method for programming an electrical stimulation system can utilize any combination of a number of aids to facilitate communication between the patient and the programmer or clinician. For example, a three dimensional model or two dimensional representation of the human body or portion of the human body can be used by the patient to indicate sites of symptoms, stimulation effects or side effects. Non-textual icons or a graphical scale can be used by the patient to respond to queries by the programmer or clinician.

### CROSS-REFERENCE TO RELATED APPLICATIONS

This application claims the benefit under 35 U.S.C. §119(e) of U.S. Provisional Patent Application Ser. No. 62/304,802, filed Mar. 7, 2016, which is incorporated herein by reference.

### FIELD

The present invention is directed to the area of implantable electrical stimulation systems and methods of making and using the systems. The present invention is also directed to systems and methods for facilitating patient-clinician communication during programming of the electrical stimulation system.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a system for identifying portions of the human body relating to a stimulation system, the system including a three dimensional model of a human body or a portion of the human body, where the model is responsive to user indication of a portion of the model, and a computer processor coupled to the model to perform the following actions: receiving the user indication of the portion of the model; and transmitting, to an external device, an indication of the portion of the model corresponding to the user indication.

In at least some embodiments, the three dimensional model is configured and arranged, in response to the user indication, to provide a visual identification on the model of the portion of the model corresponding to the user indication. In at least some embodiments, the visual indication is a lighting of the portion of the model corresponding to the user indication.

In at least some embodiments, the system further includes i) an implantable pulse generator or an external trial stimulator and ii) a stimulation lead coupled to the implantable pulse generator or external trial stimulator, wherein the actions further include requesting that the user indicates on the model where a stimulation effect from the stimulation lead is felt. In at least some embodiments, the actions further include requesting that the user indicates on the model where a symptom of a disease or disorder is felt. In at least some embodiments, the actions further include requesting that the user indicates on the model where a side effect from the stimulation lead is felt.

In at least some embodiments, the system further includes a display coupled to the processor and the actions further include displaying on the display a two dimensional representation of the human body or the portion of the human body and, in response to the user indication, visually identifying on the two dimensional representation the portion of the model corresponding to the user indication. In at least some embodiments, the actions further include receiving a user indication of a portion of the two dimensional representation; and transmitting, to an external device, an indication of the portion of the two dimensional representation corresponding to the user indication.

Another embodiment is a system for programming a stimulation system including a display; and a computer processor coupled to the display and configured and arranged to perform the following actions: receiving a direction from a programmer to present a query to a patient in response to stimulation provided to the patient via i) an implantable pulse generator or an external trial stimulator and ii) a stimulation lead coupled to the implantable pulse generator or external trial stimulator; displaying on the display a plurality of non-textual icons representing possible answers to the query; and, responsive to a selection of a one of the plurality of the non-textual icons by the patient, transmitting the selection to the programmer.

In at least some embodiments, the plurality of icons includes an icon representing an answer of “yes” and an icon representing an answer of “no”. In at least some embodiments, the plurality of icons further includes an icon representing an answer of neither “yes” nor “no”. In at least some embodiments, each of the plurality of icons is an emoticon. In at least some embodiments, each of the icons has a different color. In at least some embodiments, the color of each of the icons is associated with the answer represented by the icon.

A further embodiment is a system for programming a stimulation system including a display; and a computer processor coupled to the display and configured and arranged to perform the following actions: receiving a direction from a programmer to present a query to a patient in response to stimulation provided to the patient via i) an implantable pulse generator or an external trial stimulator and ii) a stimulation lead coupled to the implantable pulse generator or external trial stimulator; displaying on the display a graphical scale, the graphical scale including at least one non-textual indicator so that the patient can identify a meaning of relative positions along the scale; and, responsive to a selection of a position on the scale, transmitting the selection to the programmer.

In at least some embodiments, the graphical scale has a color varying along the scale. In at least some embodiments, the variation of the color is associated with the meaning of the relative positions along the scale.

In at least some embodiments, the actions further include displaying on the display a plurality of textual descriptions aligned with the scale and associated with the relative positions along the scale. In at least some embodiments, the actions further include displaying on the display a plurality of numeral values aligned with the scale. In at least some embodiments, the actions further include displaying on the display a control to halt stimulation and, upon selection of the control, transmitting to the implantable pulse generator or external trial stimulator a command to halt stimulation.

Yet other embodiments, include a computer readable medium have instructions stored thereon, where the instructions are the processor actions of any of the embodiments described above.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2 is a schematic side view of one embodiment of an electrical stimulation lead, according to the invention;

FIG. 3 is a schematic block diagram of one embodiment of a computing device, according to the invention;

FIG. 4 is a flowchart of one embodiment of a system for programming an electrical stimulation system, according to the invention;

FIG. 5 is a schematic front view of a model of a human body for use in the system of FIG. 4, according to the invention;

FIG. 6 is schematic view of a two dimensional representation of a human body for use in the system of FIG. 4, according to the invention

FIG. 7 is a flowchart of a one embodiment of a method of identifying portions of the human body relating to an electrical stimulation system, according to the invention;

FIG. 8 is a diagrammatic illustration of one embodiment of a user interface with non-textual icons, according to the invention;

FIG. 9 is a flowchart of a one embodiment of a method of programming an electrical stimulation system, according to the invention;

FIG. 10 is a diagrammatic illustration of one embodiment of a graphical scale, according to the invention; and

FIG. 11 is a flowchart of another embodiment of a method of programming an electrical stimulation system, according to the invention.

### DETAILED DESCRIPTION

The present invention is directed to the area of implantable electrical stimulation systems and methods of making and using the systems. The present invention is also directed to systems and methods for selecting stimulation parameters using targeting and steering mechanisms.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

A percutaneous lead for electrical stimulation (for example, deep brain or spinal cord stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. For illustrative purposes, the leads are described herein relative to use for deep brain stimulation, but it will be understood that any of the leads can be used for applications other than deep brain stimulation, including spinal cord stimulation, peripheral nerve stimulation, or stimulation of other nerves, muscles, and tissues.

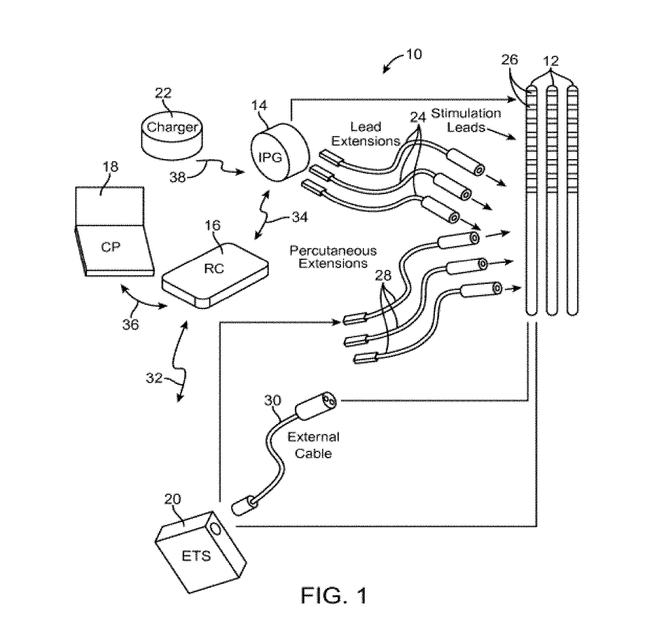

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and U.S. Pat. Nos. 7,761,165; 7,974,706; 8,175,710; 8,224,450; and 8,364,278; and U.S. Patent Application Publication No. 2007/0150036, as well as the other references cited above, all of which are incorporated by reference.

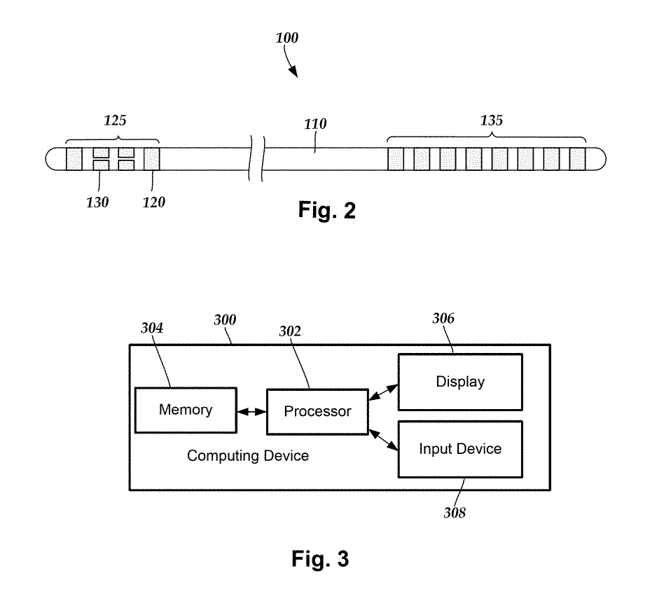

FIG. 2 illustrates one embodiment of a lead 110 with electrodes 125 disposed at least partially about a circumference of the lead 110 along a distal end portion of the lead and terminals 135 disposed along a proximal end portion of the lead. The lead 110 can be implanted near or within the desired portion of the body to be stimulated such as, for example, the brain, spinal cord, or other body organs or tissues. In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 110 can be inserted into the cranium and brain tissue with the assistance of a stylet (not shown). The lead 110 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 110, advance the lead 110, retract the lead 110, or rotate the lead 110.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 110 for deep brain stimulation can include stimulation electrodes, recording electrodes, or both. In at least some embodiments, the lead 110 is rotatable so that the stimulation electrodes can be aligned with the target neurons after the neurons have been located using the recording electrodes.

Stimulation electrodes may be disposed on the circumference of the lead 110 to stimulate the target neurons. Stimulation electrodes may be ring-shaped so that current projects from each electrode equally in every direction from the position of the electrode along a length of the lead 110. In the embodiment of FIG. 2, two of the electrodes 120 are ring electrodes 120. Ring electrodes typically do not enable stimulus current to be directed from only a limited angular range around of the lead. Segmented electrodes 130, however, can be used to direct stimulus current to a selected angular range around the lead. When segmented electrodes are used in conjunction with an implantable pulse generator that delivers constant current stimulus, current steering can be achieved to more precisely deliver the stimulus to a position around an axis of the lead (i.e., radial positioning around the axis of the lead). To achieve current steering, segmented electrodes can be utilized in addition to, or as an alternative to, ring electrodes.

The lead 100 includes a lead body 110, terminals 135, and one or more ring electrodes 120 and one or more sets of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use.

Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads may include one or more sets of segmented electrodes. Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; and 8,792,993; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; 2015/0051681; U.S. patent application Ser. Nos. 14/557,211 and 14/286,797; and U.S. Provisional Patent Application Ser. No. 62/113,291, all of which are incorporated herein by reference.

One or more electrical stimulation leads can be implanted in the body of a patient (for example, in the brain or spinal cord of the patient) and used to stimulate surrounding tissue. The lead(s) are coupled to the implantable pulse generator. After implantation, a clinician will program the implantable pulse generator using the clinician programmer, remote control, or other programming device. In at least some programming techniques, the clinician enters stimulator parameters for a stimulation program and the stimulation program is used to stimulate the patient. The clinician observes the patient response. In at least some instances, the clinician asks the patient to describe, rate, or otherwise provide information about the effects of the stimulation such as what portion of the body is affected, how strong is the stimulation effect, whether there are side effects or negative effects, and the like.

In at least some instances, the clinician may be remote from the patient. For example, the clinician may be in another room, treatment or care facility, city, state, or even country. This may be advantageous as it can allow skilled clinicians to interact with patients that are remote from the clinician without requiring travel or loss of time by the clinician. As another example, the patient may be sent home after surgery and the clinician can program the device remotely while the patient is at home.

Such remote programming, however, may encounter difficulties such as, for example, limited bandwidth, speech impaired patients, deaf patients, patients who speak a different language from the clinician, noise over the phone or video connection between patient and clinician, patient difficulty hearing due to hearing aid devices, and the like.

FIG. 3 illustrates one embodiment of a computing device 300 for use by a clinician, patient, or other person. The computing device 300 includes a processor 302 and a memory 304, a display 306, and an input device 308. The computing device 300 can be a computer, tablet, mobile device, or any other suitable device for processing information. The computing device 300 can be local to the user or can include components that are non-local to the computer including one or both of the processor 302 or memory 304 (or portions thereof). For example, in some embodiments, the user may operate a terminal that is connected to a non-local processor or memory.

The computing device 300 can utilize any suitable processor 302 including one or more hardware processors that may be local to the user or non-local to the user or other components of the computing device. The processor 302 is configured to execute instructions provided to the processor.

Any suitable memory 304 can be used for the computing device 302. The memory 304 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth™, near field communication, and other wireless media.

The display 306 can be any suitable display device, such as a monitor, screen, display, or the like, and can include a printer. The input device 308 can be, for example, a keyboard, mouse, touch screen, track ball, joystick, voice recognition system, or any combination thereof, or the like. Another input device 308 can be a camera from which the clinician can observe the patient. Yet another input device 308 is a microphone where the patient or clinician can provide responses or queries.

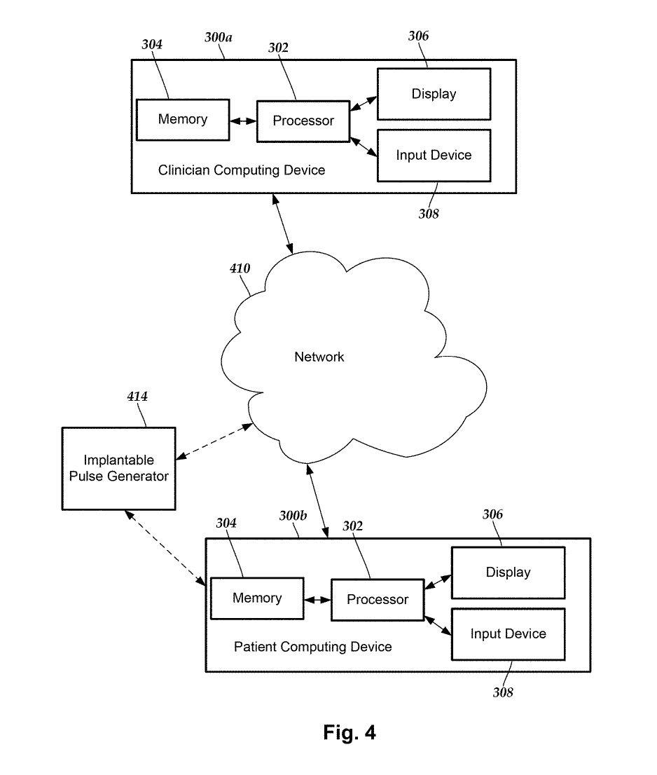

FIG. 4 illustrates one embodiment of a system for remote programming of an electrical stimulation system. The system includes a clinician computing device 300a, patient computing device 300b, a network 410, and an implantable pulse generator 414 or other programmable stimulation device. The clinician computing device 300a and patient computing device 300b can be, for example, a computing device as described above, and the two computing devices can be the same or different. In at least some embodiments, the clinician computing device 300a is a clinician programmer 18 (FIG. 1). In at least some embodiments, the patient computing device 300a is a clinician programmer 18 or remote control 16 (FIG. 1). In at least some embodiments, the patient computing device 300a can be a smartphone or tablet with a programming application running on the device. The network 410 can be any suitable type of network including, but not limited to, a local area network, a wide area network, the Internet, or any combination thereof. Although FIG. 4 illustrates the clinician computing device 300a and patient computing device 300b coupled through a network 410, it will be understood that in other embodiments, the clinician and patient computing devices 300a, 300b are directly connected and that the clinician is not necessarily located remotely from the patient.

In at least some embodiments, the clinician computing device 300a is coupled to the implantable pulse generator 414 through the network 410. In some embodiments, the clinician computing device 300a is (alternatively or additionally) coupled to the implantable pulse generator 414 via the patient computing device 300b.

Methods of communication between devices or components of a system, such as the clinician computing device 300a, the patient computing device 300b, the implantable pulse generator 414, and the network 410, can include wired (including, but not limited to, USB, mini/micro USB, HDMI, and the like) or wireless (e.g., RF, optical, infrared, near field communication (NFC), Bluetooth™, or the like) communications methods or any combination thereof. By way of further example, communication methods can be performed using any type of communication media or any combination of communication media including, but not limited to, wired media such as twisted pair, coaxial cable, fiber optics, wave guides, and other wired media and wireless media such as acoustic, RF, optical, infrared, NFC, Bluetooth™ and other wireless media.

In another possible arrangement, instead of the implantable pulse generator 414 an external trial stimulator (such as ETS 20 of FIG. 1) is to be programmed. The programming may occur when the patient is in a care facility or, if the external trial stimulator can be taken home, when the patient is in their home. Use of remote programming may improve the transition from a trial stimulation to permanent implantable pulse generator as the clinician or programmer and patient may find it easier to conduct multiple (or longer) programming sessions. The embodiments described below illustrate programming an implantable pulse generator, but it will be understood that these embodiments can also be applied to programming an external trial stimulator or any other stimulator.

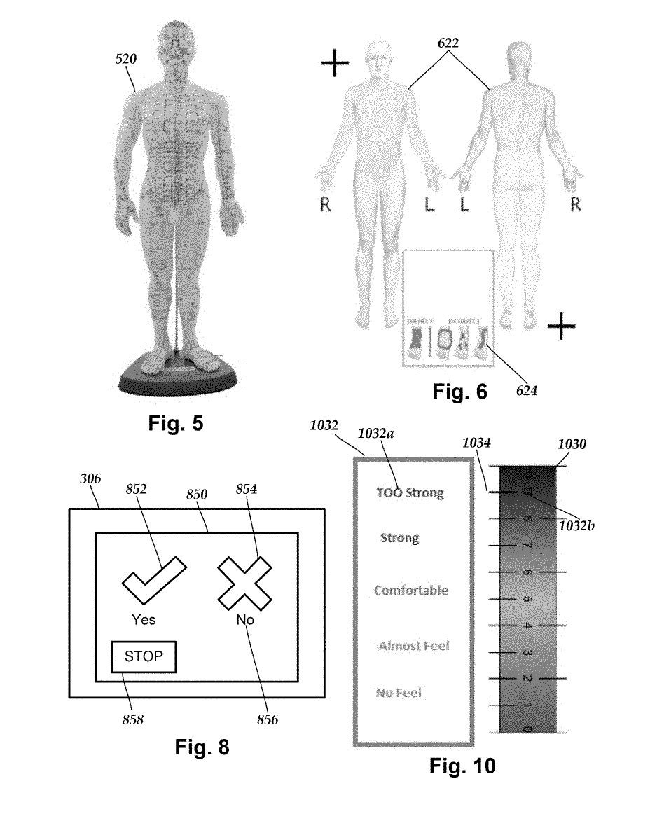

FIG. 5 illustrates an additional component that can be added to the system. A three-dimensional model 520 can be coupled to the patient computing device 300b or via the network 410 to the clinician computing device 300a. The coupling can be through wired or wireless communications. The three-dimensional model 520 can be a model of the human body or a portion of the human body.

In at least some embodiments, the three-dimensional human model 520 can be used by the patient to indicate sites of symptoms (for example, pain, trembling, seizures, or the like) by indicating the sites the model 520. In at least some embodiments, the three-dimensional human model 520 can be used by the patient to indicate sites of stimulation effects (for example, paresthesia) or side effects (for example, pain or numbness) by indicating the sites on the model 520. In some embodiments, alternatively or additionally, the clinician can indicate the sites on the model based on comments from the patient or other quantitative or qualitative observation of the patient.

The indication of the sites can be performed by touching, pointing, painting, or otherwise identifying the sites on the model. The identification of the sites can be registered by the model 520 and communicated to the clinician by wired or wireless communication. In at least some embodiments, the model 520 can be responsive to the touch, pointing, or other gestures or movements of the patient. In at least some embodiments, the model 520 may require, or be responsive to, a tool such as a stylus, pen, or pointer for registering an indication by patient.

In at least some embodiments, the portion of the model that is indicated may light up or may change color or produce some other visual distinction from other portions of the model. In some embodiments, a second indication (for example, a second touch), or actuation of an “undo” control, can negate the indication of the site of the model so that the patient can correct an error. In other embodiments, the patient or clinician may be allowed to reset the model 520 to an initial, or earlier, state in the event of an error.

In at least some embodiments, a remote programmer may have a similar model that replicates the indications or other changes made to the model 520 used by the patient. In other embodiments, the remote programmer may have a two-dimensional representation (such as that in FIG. 6) that is displayed on the display of the clinician computing device that replicates the indications or other changes made to the model 520 used by the patient. In some embodiments, the patient computing device may also display a two-dimensional representation (such as that in FIG. 6) that replicates the indication or other changes made to the model 520.

FIG. 6 illustrates a two-dimensional representation 622 of the human body or part of the human body that may be displayed on the display of the patient computing device or clinician computing device. The two-dimensional representation 622 may be used in addition to, or as an alternative to, the model 520 for the patient to indicate sites of symptoms or sites of stimulation effects or side effects. The description above with respect to manipulation and use of model 520 can also apply to the manipulation and use of the representation 622. If both model 520 and representation 622 are used together, in at least some embodiments, indications made on one of these will be replicated on the other.

FIG. 6 also includes instructions 624 for correctly (illustration 624a) and incorrectly (illustrations 624b) identifying the sites of symptoms or sites of stimulation effects or side effects. In other embodiments, icons can be used instead of the words “correct” and “incorrect”. For example, a green checkmark or a smiling face may be used instead of “correct” and a red “x” or a frowning face used instead of “incorrect”. In at least some embodiments, the clinician or patient may be allowed to select from a set of icons so that a culturally or linguistically relevant set of icons can be selected. In some embodiments, the clinician or patient may be allowed to select a language so that instructions or other words presented to the patient (or clinician) are in the desired language.

During the programming session, the clinician will typically request that the patient answer questions regarding the stimulation. In at least some instances, these questions may be binary yes/no questions. In at least some embodiments, the patient computing device can present a user interface that allows the patient to answer these questions. For example, the clinician may question whether the patient feels the stimulation or stimulation effect. The clinician may question whether the patient feels pain or other uncomfortable feelings in response to the stimulation. The clinician may question whether the patient feels the stimulation effect in the correct body region.

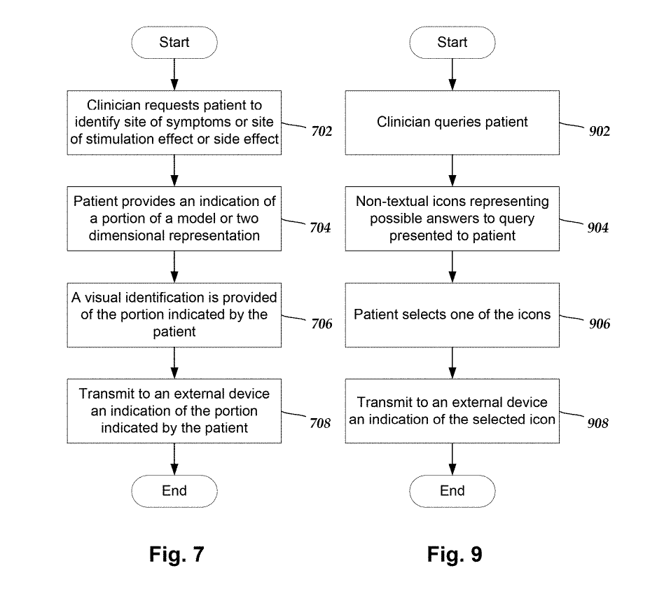

FIG. 7 is a flow chart of one method of using the model 520 or two dimensional representation 622. In step 702, the clinician, programmer, or other person asks the patient to identify or indicate a site on the model 520 or representation 622 corresponding to one or more symptoms of the disease, disorder, or condition or a site corresponding a stimulation effect or side effect. For example, the patient may be asked verbally (in person or over a telephone or other communication device) or may be asked textually (for example, on the display of the patient computing device) or both.

In step 704, the user identifies or otherwise indicates the portion of the model or two dimensional representation. Such user identification or user indication can be performed in any of the manners indicated above including touching, pointing, contacting with a stylus or other instrument, or the like.

In optional step 706, a visual indication is provided of the portion of identified or indicate by the user. The visual indication can be on the model 520 or two dimensional representation 622 that the patient used to indicate or identify the site. Alternatively or additionally, if the patient indicated or identified the site on the model 520, the visual indication may be presented on a two dimensional representation 622 provided on the display of the patient computing device.

In step 708, an indication of the portion of the model 520 or two dimensional representation 622 is transmitted to an external device, such as the clinician computing device. For example, a visual identification may be provided on a clinician model or on a two dimensional representation presented on the display of the clinician computing device.

Instead of, or in addition to, using words for the answers, the user interface can present icons that the patient can choose in order to answer the questions. FIG. 8 illustrates a display 306 with a user interface 850, icons 852, 854 disposed in the user interface, and optional text 856 corresponding to the icons. Any suitable icons can be used including emoticons or icons that have universal or cultural meaning. For example, a check mark (FIG. 8) or a smiling face might indicate “yes”, an “X” (FIG. 8), minus sign, or frowning face might indicate “no”, and, optionally, a undecided face might indicate “maybe”. As another example, a “thumbs up” or “hand forming an OK sign” icon might indicate “yes” and a “thumbs down” icon might indicate no. In addition, the icons may be colored to support their meaning using colors having a universal or cultural significance. For example, an icon signifying “yes” may be colored green, an icon signifying “no” may be colored red, and an icon signifying “maybe” or “undecided” may be colored yellow.

In addition, the user interface 850 may include a control 858 that the patient can activate to stop stimulation of the patient tissue. Alternatively or additionally, the patient computing device (or a remote control such as RC 16 of FIG. 1) can include a button or other device as an input device (such as input device 308 of patient computing device 300b of FIG. 4) that the patient can activate to stop stimulation of the patient tissue. This can be particularly useful when the clinician or programmer is programming the implantable pulse generator 414 and the programming results in uncomfortable, intense, or painful stimulation of the patient. The patient can halt the stimulation at any time using the control 858 or the separate button or other device.

FIG. 9 is a flow chart of one method of using the icons 852, 854. In step 902, the clinician, programmer, or other person presents a query to the patient. For example, the patient may be asked verbally (in person or over a telephone or other communication device) or may be asked textually (for example, on the display of the patient computing device) or both. In at least some instances, the query may be a “yes/no” question where the answers can be “yes”, “no”, and possible “maybe” or “unknown” or the like.

In step 904, non-textual icons (such as those illustrated in FIG. 8) representing possible answers to the query are presented to the patient on the display of the patient computing device. Optionally, text may also be presented with the icons.

In step 906, the patient selects at least one of the icons. Any suitable method for selection can be used, as described above.

In step 908, an indication of the selected icon is transmitted to an external device, such as the clinician computing device.

FIG. 10 illustrates another graphical scale 1030 that can be used by a patient. The graphical scale 1030 can be presented in a user interface on the display of the patient's computing device. The scale has a range of values that may or may not be numbered. The patient can use the scale to indicate, for example, the level of symptoms experienced by the patient or the level of a stimulation effect or side effect. The graphical scale includes a non-textual indicator to identify to the patient a meaning of positions along the scale. Such non-textual indicators can include the orientation of the scale, variable coloring along the scale, graduated markings along the scale, or the like or any combination thereof. In the illustrated example, the top of the scale indicates a stronger or higher level and the bottom of the scale indicates a weaker or lower level. In the case of stimulation effect, the upper portion of the scale may indicate where the stimulation effect is becoming painful or uncomfortable and the lower portion of the scale indicates where the stimulation effect is not felt or only weakly felt. The scale may be colored using a universal or culturally significant coloring scheme. For example, the scale may be colored green at the bottom followed by yellow and orange and ending with red at the top. Another color scheme might begin with blue at the bottom followed by green, yellow, orange, and red at the top. The green or blue would indicate little or no stimulation effect and the red would indicate painful or uncomfortable stimulation effect. The user interface may be arranged so that the patient can select level during or after stimulation. In some embodiments, the user interface may allow the patient to continuously or periodically indicate the level as the stimulation is changed (for example, as the stimulation amplitude is increased or decreased).

FIG. 10 also includes text 1032 that may be provided with the graphical scale 1030 to assist the patient in understanding the scale and different values or levels of the scale. The text can be, for example, textual descriptions 1032a associated with the position on the scale. Additionally or alternatively, the text can be, for example, numerical values 1032b associated with the position on the scale. In at least some embodiments, the text 1032, or portions of the text, may be colored similarly to the scale based on the position of the scale associated with the text. In addition, other markings (such as graduated markings 1034) can be presented on the graphical scale. In addition, the user interface may include a control (such as control 858 of FIG. 8) that the patient can activate to stop stimulation of the patient tissue.

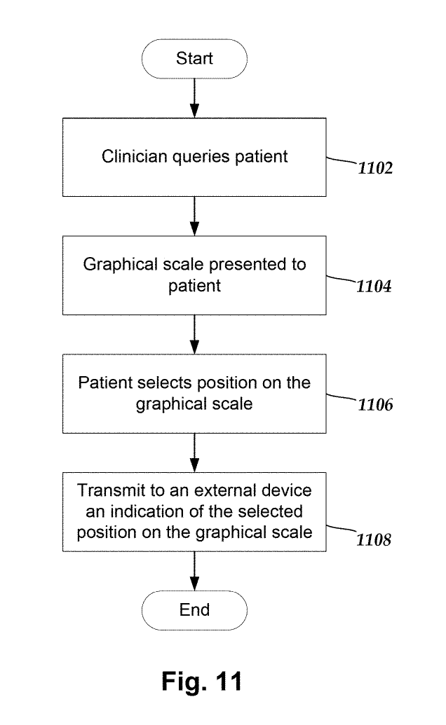

FIG. 11 is a flow chart of one method of using the graphical scale 1032. In step 1102, the clinician, programmer, or other person presents a query to the patient, such as rating the level of symptoms or the level of a stimulation effect or side effect. For example, the patient may be asked verbally (in person or over a telephone or other communication device) or may be asked textually (for example, on the display of the patient computing device) or both.

In step 1104, a graphical scale (such as that illustrated in FIG. 10) representing possible answers to the query are presented to the patient on the display of the patient computing device. Optionally, text (such as textual descriptions 1032a, numerical values 1032b, or any combination thereof) or other markings (such as graduated markings 1034) or any combination thereof may also be presented with the graphical scale.

In step 1106, the patient selects a position on the graphical scale. Any suitable method for selection can be used, as described above.

In step 1108, an indication of the selected position is transmitted to an external device, such as the clinician computing device.

Any of the methods illustrated in FIGS. 7, 9, and 11 can be then used to select stimulation electrodes and stimulation parameters based on the information identified or selected by the patient. These stimulation electrodes and stimulation parameters can be utilized to provide electrical stimulation to the patient using an implantable pulse generator and one or more leads or any other suitable stimulation system.

It will also be understood that the system, methods, and devices described with respect to FIGS. 1-11 can be used in any combination. For example, a user interface may display the icons 852, 854 of FIG. 8 with the graphical scale 1030 of FIG. 10 and the two dimensional representation 622 of FIG. 6 and be coupled to the model 520 of FIG. 5 or any other combination of these elements.

It will be understood that the system can include one or more of the methods described hereinabove with respect to FIGS. 3-11 in any combination. The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A system for identifying portions of the human body relating to a stimulation system, the system comprising:
a three dimensional model of a human body or a portion of the human body, wherein the model is responsive to user indication of a portion of the model, and
a computer processor coupled to the model and configured and arranged to perform the following actions:
receiving the user indication of the portion of the model; and
transmitting, to an external device, an indication of the portion of the model corresponding to the user indication.

2. The system of claim 1, wherein the three dimensional model is configured and arranged, in response to the user indication, to provide a visual identification on the model of the portion of the model corresponding to the user indication.

3. The system of claim 2, wherein the visual indication is a lighting of the portion of the model corresponding to the user indication.

4. The system of claim 1, further comprising i) an implantable pulse generator or an external trial stimulator and ii) a stimulation lead coupled to the implantable pulse generator or external trial stimulator, wherein the actions further comprise requesting that the user indicates on the model where a stimulation effect from the stimulation lead is felt.

5. The system of claim 4, wherein the actions further comprise requesting that the user indicates on the model where a symptom of a disease or disorder is felt.

6. The system of claim 4, wherein the actions further comprise requesting that the user indicates on the model where a side effect from the stimulation lead is felt.

7. The system of claim 1, further comprising a display coupled to the processor, wherein the actions further comprise displaying on the display a two dimensional representation of the human body or the portion of the human body and, in response to the user indication, visually identifying on the two dimensional representation the portion of the model corresponding to the user indication.

8. The system of claim 7, wherein the actions further comprise
receiving a user indication of a portion of the two dimensional representation; and
transmitting, to an external device, an indication of the portion of the two dimensional representation corresponding to the user indication.

9. A system for programming a stimulation system, the system comprising:
a display; and
a computer processor coupled to the display and configured and arranged to perform the following actions:
receiving a direction from a programmer to present a query to a patient in response to stimulation provided to the patient via i) an implantable pulse generator or an external trial stimulator and ii) a stimulation lead coupled to the implantable pulse generator or external trial stimulator;
displaying on the display a plurality of non-textual icons representing possible answers to the query; and
responsive to a selection of a one of the plurality of the non-textual icons by the patient, transmitting the selection to the programmer.

10. The system of claim 9, wherein the plurality of icons comprises an icon representing an answer of “yes” and an icon representing an answer of “no”.

11. The system of claim 10, wherein the plurality of icons further comprises an icon representing an answer of neither “yes” nor “no”.

12. The system of claim 9, wherein each of the plurality of icons is an emoticon.

13. The system of claim 9, wherein each of the icons has a different color.

14. The system of claim 13, wherein the color of each of the icons is associated with the answer represented by the icon.

15. A system for programming a stimulation system, the system comprising:
a display; and
a computer processor coupled to the display and configured and arranged to perform the following actions:
receiving a direction from a programmer to present a query to a patient in response to stimulation provided to the patient via i) an implantable pulse generator or an external trial stimulator and ii) a stimulation lead coupled to the implantable pulse generator or external trial stimulator;
displaying on the display a graphical scale, the graphical scale including at least one non-textual indicator so that the patient can identify a meaning of relative positions along the scale; and
responsive to a selection of a position on the scale, transmitting the selection to the programmer.

16. The system of claim 15, wherein the graphical scale has a color varying along the scale.

17. The system of claim 16, wherein the variation of the color is associated with the meaning of the relative positions along the scale.

18. The system of claim 15, wherein the actions further comprise displaying on the display a plurality of textual descriptions aligned with the scale and associated with the relative positions along the scale.

19. The system of claim 15, wherein the actions further comprise displaying on the display a plurality of numeral values aligned with the scale.

20. The system of claim 15, wherein the actions further comprise displaying on the display a control to halt stimulation and, upon selection of the control, transmitting to the implantable pulse generator or external trial stimulator a command to halt stimulation.
